(19) United States
(12) Patent Application Publication
(10) Pub. No.: US 2020/0275970 A1
(43) Pub. Date: Sep. 3, 2020

(54) TREATMENT DEVICE

(71) Applicant: OLYMPUS CORPORATION, Tokyo (JP)

(72) Inventor: Takayuki IDE, Tokyo (JP)

(73) Assignee: OLYMPUS CORPORATION, Tokyo (JP)

(21) Appl. No.: 16/880,254

(22) Filed: May 21, 2020

Related U.S. Application Data
(63) Continuation of application No. PCT/JP2017/044089, filed on Dec. 7, 2017.

(51) Int. Cl. A61B 18/14; A61B 18/08

(52) U.S. Cl. CPC A61B 18/1445; A61B 2017/00526; A61B 18/085

(57) ABSTRACT

A treatment device includes: a heater including a first surface and a second surface, and a heating pattern; a treatment tool bonded to the second surface and configured to apply high-frequency energy and thermal energy to a living tissue; and a flexible substrate including a first electrical conductor configured to supply high-frequency power to the treatment tool, a second electrical conductor configured to supply power to the heating pattern, and a pair of insulating layers. The first electrical conductor includes a first bonding portion exposed from the pair of insulating layers and bonded to the treatment tool, the second electrical conductor includes a second bonding portion exposed from the pair of insulating layers and bonded to the heating pattern, the treatment tool includes a projection configured to project toward the flexible substrate and surround the heater, and the first bonding portion is bonded to the projection.

This application is a continuation of International Application No. PCT/JP2017/044089, filed on Dec. 7, 2017, the entire contents of which are incorporated herein by reference.

### BACKGROUND

The present disclosure relates to a treatment device.

In the related art, there has been known a treatment device that includes an energy applying structure applying energy to a living tissue, and performs treatment (joining (or anastomosis), incising, and the like) on the living tissue by application of the energy (for example, see JP 2012-165948 A).

The energy applying structure described in JP 2012-165948 A is disposed on each of facing surfaces of a pair of jaws (first and second holding members) for gripping a living tissue. In addition, the energy applying structure includes a treatment tool (first and second high-frequency electrodes) made of a conductive material, and a heating member (electric heating chip) that is disposed on the treatment tool and generates heat when energized.

The treatment tool is directly and electrically connected to a high-frequency lead wire (high-frequency electrode energizing line) constituting a cable connected to an external energy source. Then, by supplying high-frequency power from the energy source through each high-frequency lead wire to the treatment tool of each energy applying structure disposed on each of the pair of jaws, high-frequency energy is applied to the living tissue gripped by the pair of jaws.

The heating member is electrically connected to a flexible substrate. The flexible substrate is electrically connected to a heating lead wire constituting the cable described above. The heating member then generates heat by the power supplied from the energy source through the heating lead wire and the flexible substrate, thus heating the treatment tool.

### SUMMARY

According to one aspect of the present disclosure, there is provided a treatment device including: a heater including a first surface serving as a front surface, a second surface serving as a back surface, and a heating pattern formed on the first surface and configured to generate heat when energized; a treatment tool bonded to the second surface and configured to apply high-frequency energy and thermal energy to a living tissue in contact with the treatment tool; and a flexible substrate disposed so as to face the treatment tool with the heating member interposed between the flexible substrate and the treatment tool, the flexible substrate including a first electrical conductor configured to supply high-frequency power to the treatment tool, a second electrical conductor configured to supply power to the heating pattern, and a pair of insulating layers facing each other with the first electrical conductor and the second electrical conductor interposed between the pair of insulating layers, wherein the first electrical conductor includes a first bonding portion exposed from the pair of insulating layers and bonded to the treatment tool, the second electrical conductor includes a second bonding portion exposed from the pair of insulating layers and bonded to the heating pattern, the treatment tool includes a projection configured to project toward the flexible substrate and surround the heater, and the first bonding portion is bonded to the projection.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a view schematically illustrating a treatment system according to an embodiment;

FIG. 2 is a view illustrating a gripping portion;

FIG. 3 is a view illustrating the gripping portion;

FIG. 4 is a view illustrating a first energy applying structure;

FIG. 5 is a view illustrating the first energy applying structure;

FIG. 6 is an exploded perspective view illustrating a first flexible substrate;

FIG. 7 is a partially enlarged view of the first flexible substrate;

FIG. 8A is a view for explaining a method of manufacturing the first energy applying structure;

FIG. 8B is a view for explaining the method of manufacturing the first energy applying structure;

FIG. 8C is a view for explaining the method of manufacturing the first energy applying structure;

FIG. 9 is a view illustrating a first modification of the embodiment;

FIG. 10 is a view illustrating a second modification of the embodiment;

FIG. 11 is a view illustrating a third modification of the embodiment;

FIG. 12 is a view illustrating a fourth modification of the embodiment;

FIG. 13 is a view illustrating a fifth modification of the embodiment;

FIG. 14 is a view illustrating a sixth modification of the embodiment;

FIG. 15 is a view illustrating a seventh modification of the embodiment; and

FIG. 16 is a view illustrating an eighth modification of the embodiment.

### DETAILED DESCRIPTION

Hereinafter, modes for carrying out the present disclosure (hereinafter, “embodiments”) will be described with reference to the drawings. The present disclosure is not limited by the embodiments to be described below. In addition, in the description of the drawings, the same portions are denoted by the same reference numerals.

Schematic Configuration of Treatment System

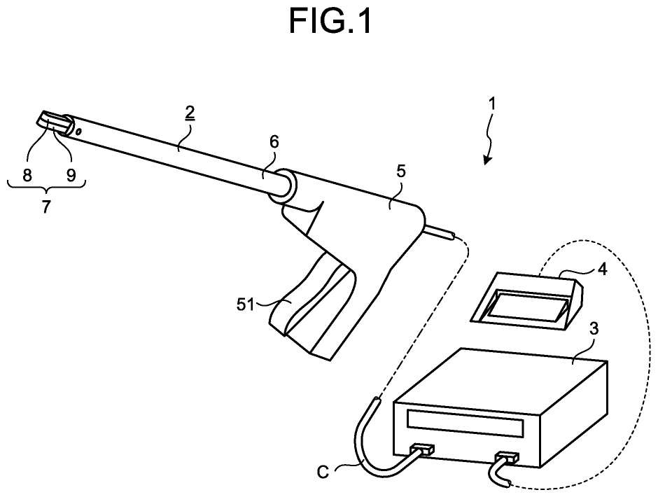

FIG. 1 is a view schematically illustrating a treatment system 1 according to the present embodiment.

The treatment system 1 applies high-frequency energy and thermal energy to a living tissue as a treatment target, thus performing treatment (joining (or anastomosis), incising, and the like) on the living tissue. As illustrated in FIG. 1, the treatment system 1 includes a treatment device 2, a control device 3, and a foot switch 4.

Configuration of Treatment Device

The treatment device 2 is, for example, a linear surgical treatment device for performing treatment on a living tissue through an abdominal wall. As illustrated in FIG. 1, the treatment device 2 includes a handle 5, a shaft 6, and a gripping portion 7.

The handle 5 is a portion that an operator holds by hand. The handle 5 includes an operation knob 51 as illustrated in FIG. 1.

As illustrated in FIG. 1, the shaft 6 has a substantially cylindrical shape and is connected to the handle 5 at one end (right end in FIG. 1). In addition, the gripping portion 7 is attached to the other end (left end in FIG. 1) of the shaft 6. Inside the shaft 6, an opening and closing mechanism (not illustrated) that opens and closes a first gripping member 8 and a second gripping member 9 (FIG. 1) constituting the gripping portion 7 according to an operation of the operation knob 51 by the operator is disposed. Further, inside the shaft 6, an electric cable C (FIG. 1) connected to the control device 3 is extended from one end side (right end side in FIG. 1) through the handle 5 to another end side (left end side in FIG. 1).

Configuration of Gripping Portion

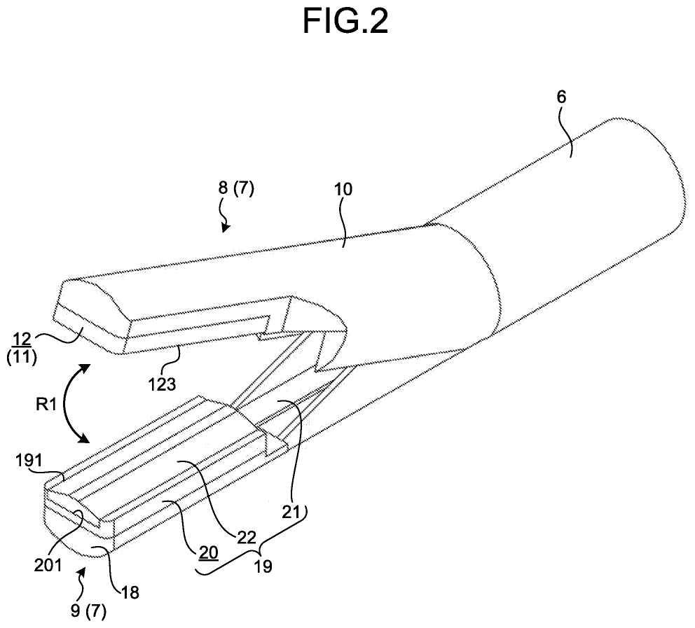

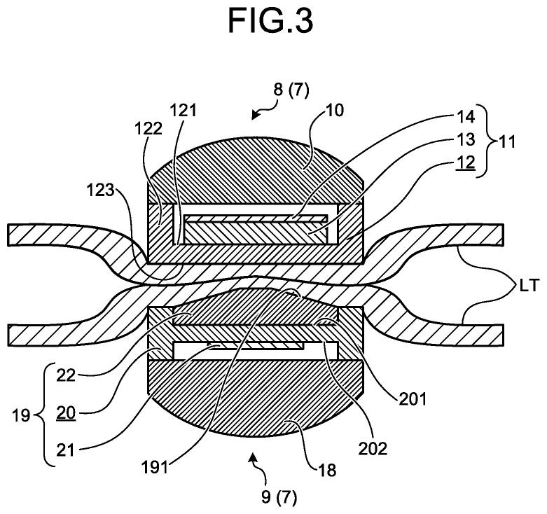

FIG. 2 and FIG. 3 are views illustrating the gripping portion 7. Specifically, FIG. 2 is a perspective view of the gripping portion 7. FIG. 3 is a cross-sectional view of the gripping portion 7 obtained by cutting the gripping portion 7 along a plane orthogonal to a longitudinal direction from a distal end to a proximal end of the gripping portion 7.

The gripping portion 7 is a portion that grips a living tissue LT (FIG. 3) such as a blood vessel and treats the living tissue LT. The gripping portion 7 includes the first gripping member 8 and the second gripping member 9 as illustrated in FIGS. 1 to 3.

The first gripping member 8 and the second gripping member 9 are supported by the other end (left end in FIG. 1 and FIG. 2) of the shaft 6 so as to be openable and closable in a direction of an arrow R1 (FIG. 2), and can grip the living tissue LT according to an operation of the operation knob 51 by an operator.

Configuration of First Gripping Member

Note that “distal end side” to be described below is the distal end side of the gripping portion 7 and means the left side in FIG. 1 and FIG. 2. In addition, “proximal end side” to be described below is the side of the shaft 6 of the gripping portion 7 and means the right side in FIG. 1 and FIG. 2.

The first gripping member 8 is disposed above the second gripping member 9 in FIGS. 1 to 3. As illustrated in FIG. 2 or FIG. 3, the first gripping member 8 includes a first jaw 10 and a first energy applying structure 11.

The first jaw 10 has an elongated shape extending in the longitudinal direction of the gripping portion 7. The first jaw 10 is rotatably supported at the proximal end side with respect to the shaft 6 to rotate, thus opening and closing with respect to the second gripping member 9.

In the present embodiment, it is configured that the second gripping member 9 is fixed to the shaft 6 and the first gripping member 8 is rotatably supported by the shaft 6, but the present disclosure is not limited thereto. For example, it may be configured that both the first gripping member 8 and the second gripping member 9 are rotatably supported by the shaft 6 to rotate, thus opening and closing. Alternatively, for example, it may be configured that the first gripping member 8 is fixed to the shaft 6, and the second gripping member 9 is rotatably supported by the shaft 6 to rotate, thus opening and closing with respect to the first gripping member 8.

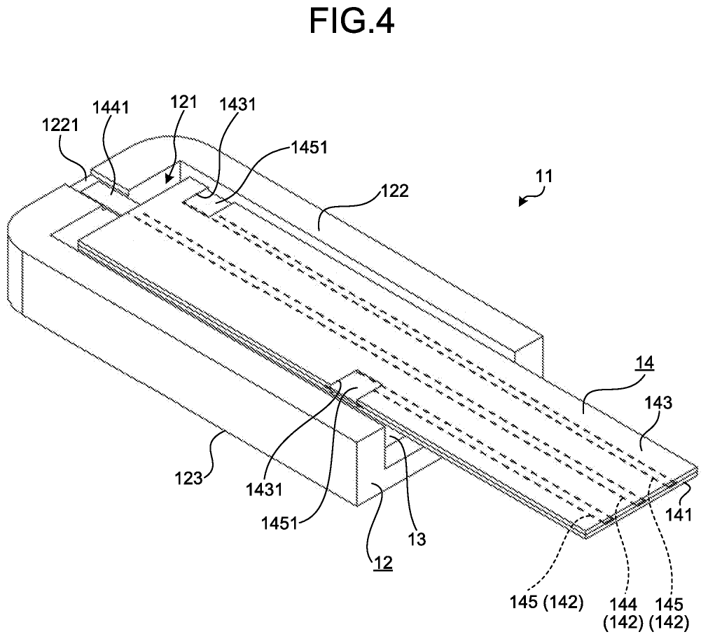

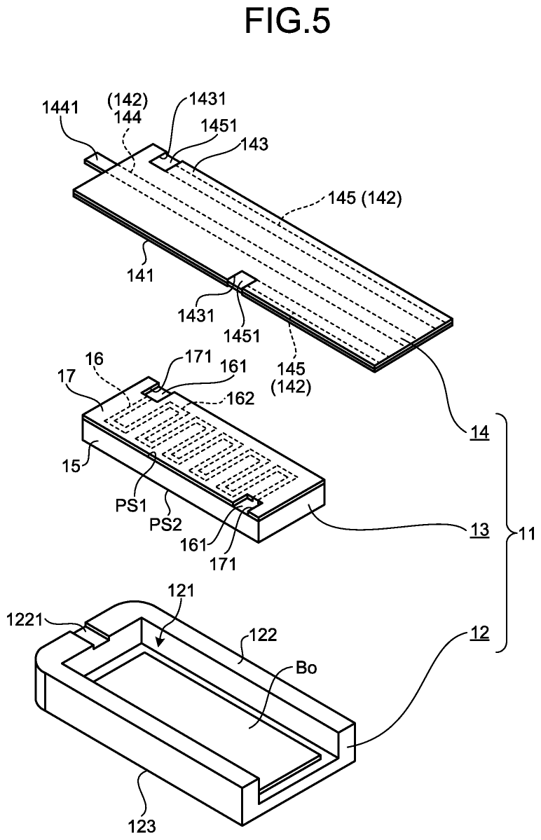

FIG. 4 and FIG. 5 are views illustrating the first energy applying structure 11. Specifically, FIG. 4 is a perspective view of the first energy applying structure 11 as viewed from above in FIGS. 1 to 3. FIG. 5 is an exploded perspective view of the first energy applying structure 11 illustrated in FIG. 4.

The first energy applying structure 11 generates high-frequency energy and thermal energy under the control of the control device 3. As illustrated in FIG. 4 or FIG. 5, the first energy applying structure 11 includes a treatment tool 12, a heating member 13, and a first flexible substrate 14.

The treatment tool 12 is made of, for example, a conductive material such as copper. In addition, as illustrated in FIG. 4 or FIG. 5, the treatment tool 12 is formed of a plate that extends in an elongated shape (elongated shape extending in longitudinal direction of gripping portion 7 (left-right direction in FIG. 4 and FIG. 5)) and has a recess 121 on one plate surface.

The recess 121 is located at the center of the treatment tool 12 in a width direction, and extends along the longitudinal direction of the treatment tool 12. Further, among side walls constituting the recess 121, the proximal end side does not include the side wall. The treatment tool 12 supports the members 13 and 14 in the recess 121, and is attached to the first jaw 10 in a posture that the recess 121 faces upward with respect to the lower surface of the first jaw 10 in FIGS. 2 and 3.

Here, in the treatment tool 12, the side wall constituting the recess 121 extends along an outer edge of the treatment tool 12, surrounds the heating member 13, and corresponds to a projection 122 (FIGS. 3 to 5) according to the present disclosure. As illustrated in FIG. 4 or FIG. 5, a bonding groove 1221 to which a high-frequency pad 1441 of the first flexible substrate 14 is bonded is formed on a distal end side of a projecting end of the projection 122. Further, in the treatment tool 12, the plate surface on which the recess 121 is not formed is constituted by a flat surface orthogonal to a thickness direction of the treatment tool 12 (vertical direction in FIGS. 3 to 5), and functions as a first gripping surface 123 gripping the living tissue LT with the second gripping member 9.

The heating member 13 has an outer size slightly smaller than the inner size of the recess 121 and is bonded to a bottom surface of the recess 121. The heating member 13 has a resistance pattern 162 that generates heat when energized, and heats the treatment tool 12 by the heat of the resistance pattern 162. As illustrated in FIG. 5, the heating member 13 includes an insulating member 15, a heating pattern 16, and a cover layer 17.

The insulating member 15 is made of, for example, an insulating material having high thermal conductivity, such as alumina or aluminum nitride, and transmits the heat of the resistance pattern 162 to the treatment tool 12. Further, as illustrated in FIG. 5, the insulating member 15 is formed of an elongated plate extending in the longitudinal direction of the gripping portion 7.

Here, in the insulating member 15, one plate surface (upper plate surface in FIG. 5) corresponds to a first surface PS1 (FIG. 5) according to the present disclosure. Further, in the insulating member 15, the other plate surface (lower plate surface in FIG. 5) corresponds to a second surface PS2 (FIG. 5) according to the present disclosure. The heating member 13 has the second surface PS2 bonded to the bottom surface of the recess 121.

The heating pattern 16 is obtained by processing a platinum thin film, and includes a pair of heating connectors 161 and the resistance pattern 162 as illustrated in FIG. 5. The heating pattern 16 is formed by patterning a platinum thin film that is formed on the first surface PS1 by vapor deposition, sputtering, or the like using photolithography.

The material of the heating pattern 16 is not limited to the platinum thin film, and may be a conductive thin film material such as nickel or titanium. The heating pattern 16 is not limited to the configuration in which a thin film is patterned on the first surface PS1, and a configuration in which a thick film paste material such as ruthenium oxide is formed on the first surface PS1 by a printing technique may be employed.

The pair of heating connectors 161 have a layer structure of an adhesion layer inserted between the insulating members 15 and the heating connector 161 as needed, an adhesion layer added to a surface side, and a protective layer. Further, as illustrated in FIG. 5, the pair of heating connectors 161 are located at diagonal positions on the first surface PS1, that is, at corner portions on the distal end side (left end side in FIG. 5) and the proximal end side (right end side in FIG. 5). Then, a pair of heating conductive lines 145 constituting the first flexible substrate 14 are bonded (connected) to the pair of heating connectors 161, respectively.

The resistance pattern 162 is connected (conductive) to the heating connector 161 on the proximal end side at one end, extends while meandering in a wave shape toward the distal end side, and is connected (conductive) to the heating connector 161 on the distal end side at the other end. Then, the resistance pattern 162 generates heat when a voltage is applied (energized) to the pair of heating connectors 161 through the pair of heating conductive lines 145 under the control of the control device 3.

The cover layer 17 is made of, for example, an insulating material such as polyimide having high thermal conductivity. The cover layer 17 is formed of an elongated plate (elongated plate extending in longitudinal direction of gripping portion 7) having the same width and length dimensions as the insulating member 15. The cover layer 17 has one plate surface (lower plate surface in FIG. 5) bonded to the first surface PS1 to cover the heating pattern 16. The cover layer 17 includes apertures 171 (FIG. 5) that pass the cover layer 17 and expose the pair of heating connectors 161 to outside at positions facing the pair of heating connectors 161.

In the present embodiment, it is desirable that the thermal resistance of the cover layer 17 is higher than the thermal resistance of the insulating member 15. The cover layer 17 may be made of the same material as the insulating member 15. In this case, if the thickness dimension of the cover layer 17 is made larger than the thickness dimension of the insulating member 15, the thermal resistance of the cover layer 17 can be made larger than the thermal resistance of the insulating member 15. By making the thermal resistance of the cover layer 17 larger than the thermal resistance of the insulating member 15 as described above, more heat generated in the resistance pattern 162 can be transmitted to a side of the insulating member 15.

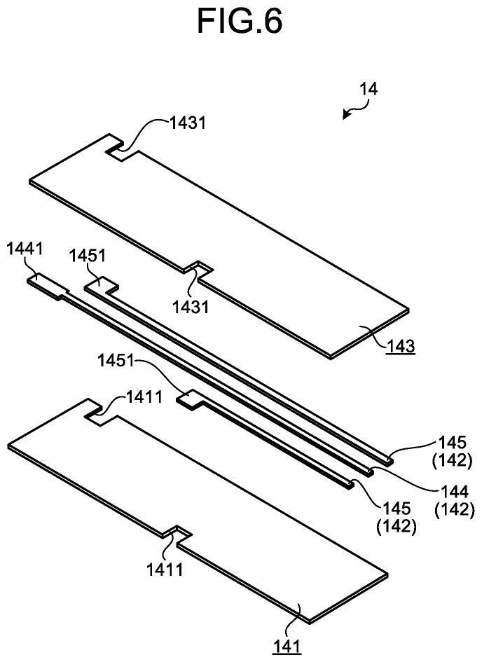

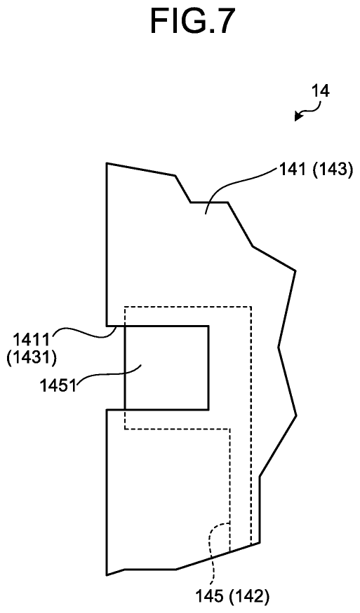

FIG. 6 is an exploded perspective view illustrating the first flexible substrate 14. FIG. 7 is a partially enlarged view of the first flexible substrate 14.

The first flexible substrate 14 corresponds to a flexible substrate according to the present disclosure. The first flexible substrate 14 is electrically connected to the electric cable C extended from one end side to the other end side of the shaft 6, the treatment tool 12, and the heating member 13, respectively, and relays the electric cable C to the treatment tool 12 and the heating member 13. The first flexible substrate 14 is disposed so as to face the first surface PS1. The first flexible substrate 14 includes a first insulating layer 141, a conductive layer 142, and a second insulating layer 143, as illustrated in FIGS. 4 to 6.

The first insulating layer 141 corresponds to an insulating layer according to the present disclosure. The first insulating layer 141 is an elongated sheet (elongated sheet extending in longitudinal direction of the gripping portion 7) made of an insulating material such as polyimide, and has a width dimension slightly smaller than the width dimension of the recess 121 and a length dimension larger than the length dimension of the recess 121 in the longitudinal direction.

The conductive layer 142 is made of a rolled copper foil, and is formed on one surface (upper surface in FIG. 6) of the first insulating layer 141. As illustrated in FIGS. 4 to 6, the conductive layer 142 includes a high-frequency conductive line 144 and a pair of heating conductive lines 145.

The high-frequency conductive line 144 corresponds to a first electrical conductor according to the present disclosure. As illustrated in FIGS. 4 to 6, the high-frequency conductive line 144 is located at the center in the width direction on one surface of the first insulating layer 141, and extends linearly from its proximal end to its distal end. A first high-frequency lead wire (not illustrated) constituting the electric cable C is connected (bonded) to an end of the high-frequency conductive line 144 on the proximal end side. In addition, as illustrated in FIGS. 4 to 6, the high-frequency pad 1441 that has a rectangular shape in a planar view, projects from an outer edge of the first insulating layer 141 to the distal end side, and is bonded to the treatment tool 12 (bonding groove 1221) is disposed at the distal end of the high-frequency conductive line 144. The high-frequency pad 1441 corresponds to a first bonding portion according to the present disclosure.

The pair of heating conductive lines 145 each correspond to a second electrical conductor according to the present disclosure. As illustrated in FIGS. 4 to 6, the pair of heating conductive lines 145 are located on both sides in the width direction on one surface of the first insulating layer 141 with the high-frequency conductive line 144 interposed therebetween. Each of the pair of heating conductive lines 145 extends from the proximal end to the position facing each of the pair of heating connectors 161. A pair of heating lead wires (not illustrated) constituting the electric cable C are respectively connected (bonded) to the ends of the pair of heating conductive lines 145 on the proximal end side. As illustrated in FIGS. 4 to 6, a heating pad 1451 that has a rectangular shape in a planar view and is bonded to each of the pair of heating connectors 161 is disposed at each of the distal ends of the pair of heating conductive lines 145. Each heating pad 1451 corresponds to a second bonding portion according to the present disclosure.

The second insulating layer 143 corresponds to an insulating layer according to the present disclosure. The second insulating layer 143 is a sheet made of the same material as the first insulating layer 141 and having the same shape. The second insulating layer 143 is bonded to one surface of the first insulating layer 141 and covers the conductive layer 142.

Cut-away portions 1411, 1431 that respectively pass the first and second insulating layers 141, 143 and expose parts of the pair of heating pads 1451 to the outside are formed at positions facing the pair of heating pads 1451 on the first and second insulating layers 141, 143, as illustrated in FIGS. 5 to 7. More specifically, the cut-away portions 1411, 1431 have a rectangular shape in a planar view. As illustrated in FIG. 7, the pair of heating pads 1451 are exposed to the outside via the cut-away portions 1411, 1431 such that among four sides of the rectangular shape in a planar view, only one side on an outer edge side is located in the cut-away portions 1411, 1431, and the other three sides are sandwiched between the first insulating layer 141 and the second insulating layer 143. The cut-away portions 1411, 1431 each correspond to an exposing aperture according to the present disclosure.

Method of Manufacturing First Energy Applying Structure

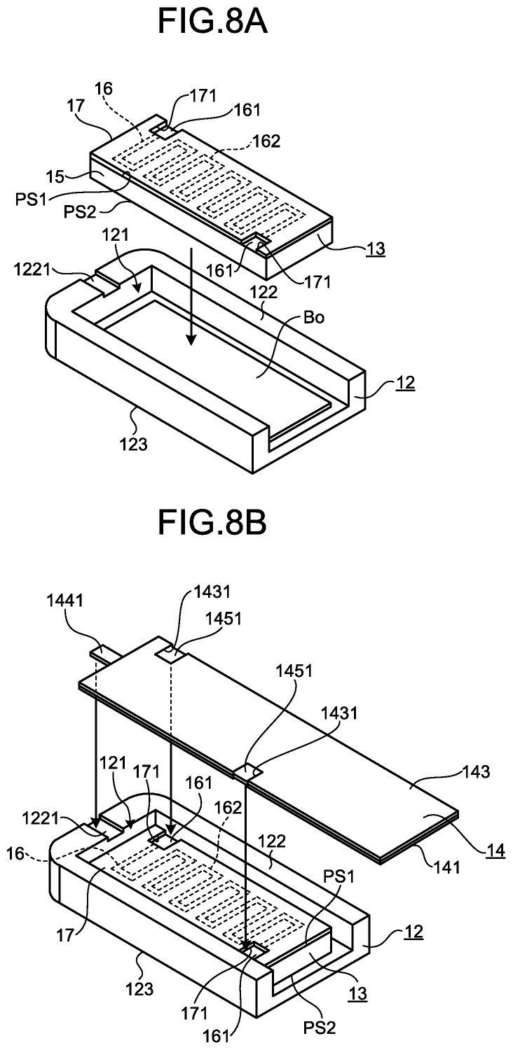

FIGS. 8A to 8C are views for explaining a method of manufacturing the first energy applying structure 11.

Next, the method of manufacturing the first energy applying structure 11 described above (method of manufacturing treatment device according to the present disclosure) will be described with reference to FIGS. 8A to 8C.

First, as illustrated in FIG. 8A, a worker disposes a bonding material Bo on the bottom surface of the recess 121, and houses the heating member 13 in the recess 121 in a posture that the second surface PS2 faces the bottom surface. At this time, in order to increase the positional accuracy between the treatment tool 12 and the heating member 13, it is desirable to use a positioning jig or to form a positioning marker, a positioning groove, and a displacement prevention groove in the treatment tool 12 in advance. Then, the worker cures the bonding material Bo while applying an appropriate load between the treatment tool 12 and the heating member 13, and bonds the treatment tool 12 to the heating member 13 (first bonding step).

Examples of the bonding material Bo include bonding materials having high thermal conductivity such as a solder, a conductive paste, and a high thermal conductive adhesive sheet.

Next, as illustrated in FIG. 8B, the worker causes the first flexible substrate 14 to face the treatment tool 12 with the heating member 13 interposed therebetween, and stacks the bonding groove 1221 and the high-frequency pad 1441, and the pair of heating connectors 161 and the pair of heating pads 1451 in a thickness direction of the first flexible substrate 14 (vertical direction in FIG. 8B). At this time, the positional accuracy between the treatment tool 12 and the heating member 13 is achieved at the first bonding step, the heating pattern 16 is formed by a photolithographic technique and thus has high shape accuracy, and the high-frequency pad 1441 and the pair of heating pads 1451 are formed on the single first flexible substrate 14 and thus the position accuracy required for manufacturing the first flexible substrate 14 is achieved. Consequently, the positioning of the bonding groove 1221 and the high-frequency pad 1441, and of the pair of heating connectors 161 and the pair of heating pads 1451 can be easily performed.

Next, the worker brings a probe Pr (FIG. 8C) of a resistance welding machine or an ultrasonic welding machine into contact with the high-frequency pad 1441 from a side of the first flexible substrate 14, applies bonding energy from the probe Pr to perform spot welding. As a result, the high-frequency pad 1441 is bonded to the bonding groove 1221 (second bonding step). Similarly, the worker sequentially brings the probe Pr into contact with the pair of heating pads 1451 from the side of the first flexible substrate 14, and applies the bonding energy from the probe Pr to perform spot welding. As a result, the pair of heating pads 1451 are sequentially bonded to the pair of heating connectors 161 (third bonding step).

With the first to third bonding steps described above, the first energy applying structure 11 is manufactured.

Configuration of Second Gripping Member

As illustrated in FIG. 2 or FIG. 3, the second gripping member 9 includes a second jaw 18 and a second energy applying structure 19.

The second jaw 18 is a portion obtained by extending a part of the shaft 6 toward the distal end side, and is formed in an elongated shape extending in the longitudinal direction of the gripping portion 7.

The second energy applying structure 19 generates high-frequency energy under the control of the control device 3. As illustrated in FIG. 2 or FIG. 3, the second energy applying structure 19 includes a counter electrode 20, a second flexible substrate 21, and a counter member 22.

The counter electrode 20 is made of, for example, a conductive material such as copper. Further, the counter electrode 20 is formed of an elongated plate (elongated plate extending in longitudinal direction of gripping portion 7) having recesses 201, 202 (FIG. 3) on both plate surfaces.

The recess 201 is located at the center in the width direction on the upper plate surface of the counter electrode 20 in FIG. 2 and FIG. 3, and extends along a longitudinal direction of the counter electrode 20.

Further, among side walls constituting the recess 201, the distal end side and the proximal end side do not include the side wall.

The recess 202 is located at the center in the width direction on the lower plate surface of the counter electrode 20 in FIG. 2 and FIG. 3, and extends along the longitudinal direction of the counter electrode 20.

Further, among side walls constituting the recess 202, the proximal end side does not include the side wall.

The counter electrode 20 is attached to the upper surface of the second jaw 18 in FIG. 2 and FIG. 3 in a posture that the recess 201 faces upward.

The second flexible substrate 21 is fixed to a bottom surface of the recess 202 at its distal end side, electrically connected to a second high-frequency lead wire (not illustrated) of the electric cable C extended from one end side to the other end side of the shaft 6 and the counter electrode 20, and relays the second high-frequency lead wire to the counter electrode 20.

The counter member 22 is made of an insulating material. The counter member 22 has an outer size that is substantially the same as the inner size of the recess 201, and is fitted into the recess 201. In the counter member 22, the upper surface in FIG. 2 and FIG. 3 projects upward toward the center in the width direction, and the counter member 22 thus has a substantially mountain-like shape whose projecting end is formed in a flat shape orthogonal to a thickness direction (vertical direction in FIG. 3) of the counter member 22.

In the second energy applying structure 19, the upper surface in FIG. 2 and FIG. 3 functions as a second gripping surface 191 that grips the living tissue LT with the first gripping surface 123.

Configuration of Control Device and Foot Switch

The foot switch 4 is a portion operated by an operator with his foot. In response to the operation on the foot switch 4, the control device 3 switches on and off the power supply to the treatment device 2.

The means for switching on and off is not limited to the foot switch 4, and a switch operated by hand or the like may be used.

The control device 3 is configured by including a CPU (Central Processing Unit) and the like, and totally controls the operation of the treatment device 2 according to a predetermined control program. More specifically, the control device 3 supplies high-frequency power to the treatment tool 12 and the counter electrode 20 through the electric cable C (first and second high-frequency lead wires), the high-frequency conductive line 144, and the second flexible substrate 21, in response to the operation of the foot switch 4 by the operator (operation of turning on power). In addition, the control device 3 applies a voltage (supplies power) to the pair of heating connectors 161 through the electric cable C (a pair of heating lead wires) and the pair of heating conductive lines 145.

Operation of Treatment System

Next, an operation of the treatment system 1 described above will be described.

An operator holds the treatment device 2 by hand and inserts the distal end portion (gripping portion 7 and part of shaft 6) of the treatment device 2 into an abdominal cavity through an abdominal wall using, for example, a trocar. The operator then operates the operation knob 51 and grips the living tissue LT as a treatment target at the gripping portion 7 (with first gripping surface 123 and second gripping surface 191).

Next, the operator operates the foot switch 4 to switch on the power supply from the control device 3 to the treatment device 2. When the power supply is switched on, the control device 3 supplies high-frequency power to the treatment tool 12 and the counter electrode 20 through the electric cable C (first and second high-frequency lead wires), the high-frequency conductive line 144, and the second flexible substrate 21. That is, high-frequency energy is applied to the living tissue LT gripped by the first gripping surface 123 and the second gripping surface 191 and degeneration occurs with the high-frequency energy. The living tissues LT are then joined to each other. Further, the control device 3 applies a voltage to the pair of heating connectors 161 through the electric cable C (a pair of heating lead wires) and the pair of heating conductive lines 145 to heat the resistance pattern 162. The heat from the resistance pattern 162 is transmitted to the treatment tool 12 through the insulating member 15 and the bonding material Bo. The temperature of the living tissue LT that is in contact with the treatment tool 12 (first gripping surface 123) increases due to the heat (application of thermal energy) of the treatment tool 12, and the living tissue LT is incised by both effects of extreme degeneration due to the temperature increase and pressing by the gripping portion 7.

In the above description, a case where high-frequency energy is used to join the living tissues LT and thermal energy is used to incise the living tissue LT has been described. However, by appropriately adjusting the thermal energy, the thermal energy can cooperate with the high-frequency energy to achieve stronger joining and faster incision.

The present embodiment described above achieves the following effects.

The first energy applying structure 11 according to the present embodiment is manufactured by the first to third bonding steps described above. That is, since the high-frequency wire (high-frequency conductive line 144) and the heating wire (heating conductive line 145) are disposed on the same first flexible substrate 14, the wiring is compact and the positioning of each wire and a bonding target (bonding groove 1221 and heating connector 161) is easily performed. As a result, the bonding steps (second and third bonding steps) of the respective wires can be simplified and the assemblability of the first energy applying structure 11 (treatment device 2) can be improved.

Further, it is not necessary to have a space for preventing interference between the wires. In particular, by forming the wires as the conductive layer 142 disposed on the first flexible substrate 14, the thickness of the first energy applying structure 11 can be reduced as compared with a case where wiring is performed with lead wires. For this reason, the first energy applying structure 11 (treatment device 2) can be made compact.

As described above, the method of manufacturing the treatment device 2 and the treatment device 2 according to the present embodiment achieve the effect that the assemblability is improved while compactness is achieved.

In the first energy applying structure 11 according to the present embodiment, the high-frequency conductive line 144 has the high-frequency pad 1441 projecting from the outer edges of the first and second insulating layers 141, 143. At the second bonding step, the first flexible substrate 14 faces the treatment tool 12 with the heating member 13 interposed therebetween, and the high-frequency pad 1441 and the bonding groove 1221 are stacked in the thickness direction of the first flexible substrate 14. In such a state, spot welding is performed from the side of the first flexible substrate 14, so that the high-frequency pad 1441 and the bonding groove 1221 are bonded to each other. That is, the bonding area of the high-frequency pad 1441 and the bonding groove 1221 can be made into a small area subjected to spot welding in order to cope with the compactness of the first energy applying structure 11 (treatment device 2), and at the same time, bonding can be performed with high strength. Further, since the high-frequency pad 1441 projects from the outer edges of the first and second insulating layers 141, 143, the bonding energy can be directly applied from the probe Pr to the high-frequency pad 1441, and the high-frequency pad 1441 can be stably bonded to the bonding groove 1221.

In the first energy applying structure 11 according to the present embodiment, the first insulating layer 141 and the second insulating layer 143 includes the cut-away portions 1411 and 1431 that expose the heating pads 1451 to the outside, respectively. At the third bonding step, the first flexible substrate 14 faces the treatment tool 12 with the heating member 13 interposed therebetween, and the heating pad 1451 and the heating connector 161 are stacked in the thickness direction of the first flexible substrate 14. In such a state, spot welding is performed from the side of the first flexible substrate 14, so that the heating pad 1451 and the heating connector 161 are bonded to each other. That is, the bonding area of the heating pad 1451 and the heating connector 161 can be made into a small area subjected to spot welding in order to cope with the compactness of the first energy applying structure 11 (treatment device 2), and at the same time, bonding can be performed with high strength. Further, since the heating pad 1451 is exposed to the outside through the cut-away portions 1411, 1431, the bonding energy can be directly applied from the probe Pr to the heating pad 1451, and the heating pad 1451 can be stably bonded to the heating connector 161.

Furthermore, at the second and third bonding steps, the directions that the probe Pr is brought into contact with the high-frequency pad 1441 and the heating pad 1451 are the same. For this reason, the second and third bonding steps can be simplified, and the assemblability can be further improved.

Meanwhile, in the heating member 13, the resistance pattern 162 contributes to actual heating, and the pair of heating connectors 161 are non-heating portions that do not generate heat. In the present embodiment, the pair of heating connectors 161 are located at diagonal positions on the first surface PS1, that is, at corner portions on the distal end side and the proximal end side. That is, as the pair of heating connectors 161 that are non-heating portions are disposed to be separated from each other, the thermal uniformity of the heating member 13 can be improved.

In addition, in order to stably bond the heating pad 1451 to the heating connector 161 with high strength, it is necessary for the heating connector 161 to have a certain area. In the present embodiment, the pair of heating connectors 161 are disposed on the first surface PS1 so as to be separated from each other in the longitudinal direction. Consequently, the area of the heating connector 161 can be set to be larger than that in a configuration in which the pair of heating connectors 161 are arranged in the width direction.

In the first energy applying structure 11 according to the present embodiment, the heating pad 1451 is exposed to the outside via the cut-away portions 1411, 1431 such that among four sides of the rectangular shape in a planar view, only one side is located in the cut-away portions 1411, 1431, and the other three sides are sandwiched between the first insulating layer 141 and the second insulating layer 143. That is, by supporting many sides of the heating pad 1451, the strength of the heating pad 1451 can be improved.

### OTHER EMBODIMENTS

The mode for carrying out the present disclosure has been described above, but the present disclosure should not be limited only by the embodiment described above.

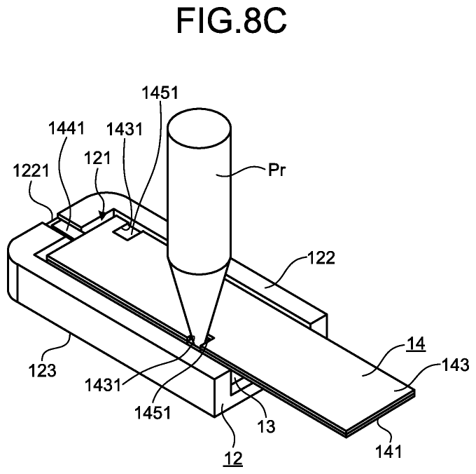

FIG. 9 is a view illustrating a first modification of the present embodiment.

In the embodiment described above, a first energy applying structure 11A according to the present first modification illustrated in FIG. 9 may be used instead of the first energy applying structure 11.

As illustrated in FIG. 9, the first energy applying structure 11A is different from the first energy applying structure 11 described in the above embodiment in that an insulating resin member Re is added.

Specifically, the first energy applying structure 11A is manufactured by sealing the pair of heating pads 1451 exposed to outside with the resin member Re so as to cover the pair of heating pads 1451 (sealing step) after the first to third bonding steps described in the above embodiment.

Here, it is desirable to not only apply the resin member Re on a surface of the first flexible substrate 14 but also cause the resin member Re to permeate a side surface of the first flexible substrate 14 and an interface between the first flexible substrate 14 and the heating member 13, in order to completely seal the pair of heating pads 1451. Consequently, a resin member having a relatively low viscosity before curing may be used as the resin member Re so as to easily permeate the interface. Alternatively, two types of resin members may be used as the resin member Re. For example, a low-viscosity resin member may be used for the interface and a high-viscosity resin member may be used for the surface and side surface of the first flexible substrate 14 so as to prevent sagging. Examples of such a resin member include a silicone resin, an epoxy resin, and a polyimide resin.

According to the first modification described above, in addition to effects similar to those of the embodiment described above, the pair of heating pads 1451 can be reliably insulated and the power for heating can be prevented from leaking to the outside.

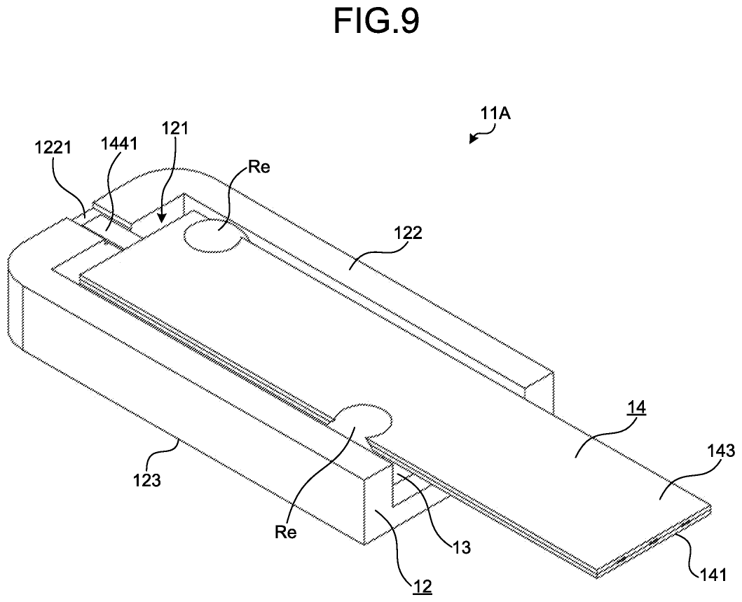

FIG. 10 is a view illustrating a second modification of the present embodiment.

In the embodiment described above, a first energy applying structure 11B according to the present second modification illustrated in FIG. 10 may be used instead of the first energy applying structure 11.

As illustrated in FIG. 10, the first energy applying structure 11B is different from the first energy applying structure 11 described in the above embodiment in that a treatment tool 12B and a first flexible substrate 14B are used instead of the treatment tool 12 and the first flexible substrate 14.

As illustrated in FIG. 10, the treatment tool 12B is different from the treatment tool 12 described in the above embodiment in that the projection 122 is not formed.

As illustrated in FIG. 10, the first flexible substrate 14B is different from the first flexible substrate 14 described in the above embodiment in that a high-frequency pad 1441B having a shape different from that of the high-frequency pad 1441 is used.

As illustrated in FIG. 10, the high-frequency pad 1441B includes a connecting portion 1442 projecting from outer edges of the first and second insulating layers 141, 143 to a side of the treatment tool 12 and a bonding portion main body 1443 bent substantially at a right angle from the connecting portion 1442 along a back surface of the first gripping surface 123 in the treatment tool 12B. Similarly to the second bonding step described in the above embodiment, bonding energy is applied from the probe Pr to the bonding portion main body 1443, and the high-frequency pad 1441B is bonded to the back surface of the first gripping surface 123 in the treatment tool 12B by spot welding.

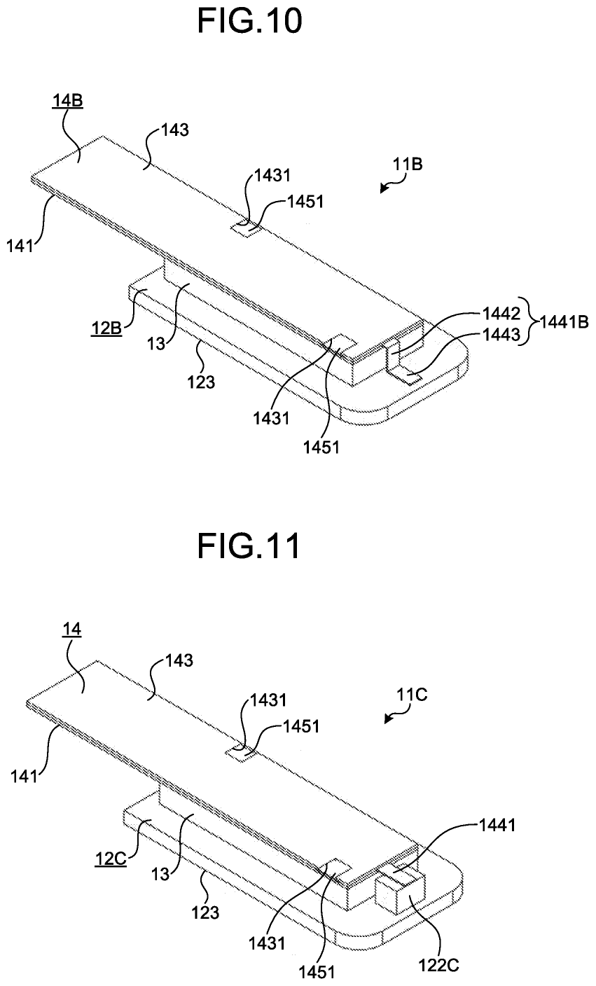

FIG. 11 is a view illustrating a third modification of the present embodiment.

In the embodiment described above, a first energy applying structure 11C according to the present third modification illustrated in FIG. 11 may be used instead of the first energy applying structure 11.

As illustrated in FIG. 11, the first energy applying structure 11C is different from the first energy applying structure 11 described in the above embodiment in that a treatment tool 12C is used instead of the treatment tool 12.

The treatment tool 12C does not have the projection 122 as in the treatment tool 12B described in the second modification. In the treatment tool 12C, a projection 122C projecting toward the first flexible substrate 14 is disposed at a position facing the high-frequency pad 1441 on a back surface of the first gripping surface 123. Similarly to the second bonding step described in the above embodiment, bonding energy is applied from the probe Pr to the high-frequency pad 1441, and the high-frequency pad 1441 is bonded to the projection 122C by spot welding.

According to the second and third modifications described above, in addition to effects similar to those of the embodiment described above, it is possible to improve the degree of freedom of the shape of the treatment tools 12B, 12C.

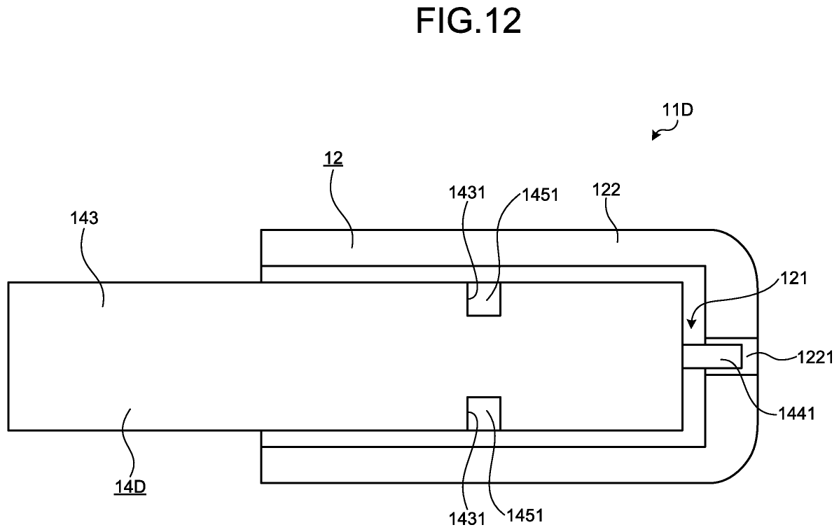

FIG. 12 is a view illustrating a fourth modification of the present embodiment.

In the embodiment described above, a first energy applying structure 11D according to the present fourth modification illustrated in FIG. 12 may be used instead of the first energy applying structure 11.

As illustrated in FIG. 12, the first energy applying structure 11D is different from the first energy applying structure 11 described in the above embodiment in positions where the pair of heating pads 1451 (a pair of cut-away portions 1411, 1431 and a pair of heating connectors 161) are formed.

Specifically, in the first energy applying structure 11D according to the fourth modification, the pair of heating pads 1451 (a pair of cut-away portions 1411, 1431 and a pair of heating connectors 161) are disposed so as to face to each other in a width direction at a substantially central position in the longitudinal direction of the heating member 13, as illustrated in FIG. 12.

The fourth modification described above achieves the following effect in addition to effects similar to those of the embodiment described above.

Meanwhile, when the resistance pattern 162 is formed over the entire first surface PS1, the temperature distribution of the first surface PS1 tends to be high at the central position and low at the outer edge. In the present fourth modification, the pair of heating connectors 161 are disposed at a substantially central position in the longitudinal direction of the heating member 13 (first surface PS1). That is, by setting the pair of heating connectors 161 that are non-heating portions at the position where the temperature is highest in the temperature distribution described above, the temperature distribution described above can be smoothed and the thermal uniformity of the heating member 13 can be improved.

Further, it is assumed in the fourth modification that the pair of heating pads 1451 are sealed with the resin member Re at the sealing step as described in the first modification. In this case, since the pair of heating pads 1451 are disposed close to each other, the pair of heating pads 1451 can be sealed at the same time and the sealing step can be simplified.

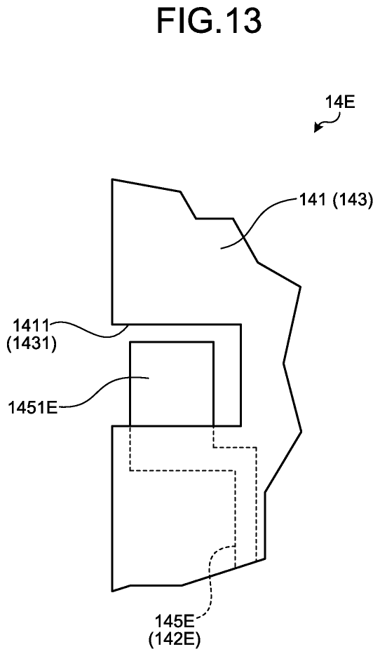

FIG. 13 is a view illustrating a fifth modification of the present embodiment. Specifically, FIG. 13 is a view corresponding to FIG. 7.

In the embodiment described above, a heating pad 1451E (heating conductive line 145E, conductive layer 142E, and first flexible substrate 14E) according to the fifth modification illustrated in FIG. 13 may be used instead of the heating pad 1451.

Specifically, as illustrated in FIG. 13, the heating pad 1451E is exposed to outside via the cut-away portions 1411, 1431 such that among four sides of the rectangular shape in a planar view, three sides are located in the cut-away portions 1411, 1431, and the remaining one side is sandwiched between the first insulating layer 141 and the second insulating layer 143. That is, the heating pad 1451E can be elastically deformed in a thickness direction of the first flexible substrate 14E (direction orthogonal to plane of FIG. 13) with the edges of the cut-away portions 1411, 1431 as fulcrums.

The fifth modification described above achieves the following effect in addition to effects similar to those of the embodiment described above.

Meanwhile, there is a gap corresponding to the thickness of the cover layer 17 and the first insulating layer 141 between the heating pad 1451E and the heating connector 161. For this reason, when the heating pad 1451E is bonded to the heating connector 161, it is necessary to forcibly deform the heating pad 1451E so as to fill the gap. In the fifth modification, the heating pad 1451E can be elastically deformed in the thickness direction of the first flexible substrate 14E. Consequently, the heating pad 1451E is deformed so as to fill the gap described above, so that the heating pad 1451E can be easily bonded to the heating connector 161.

The shape of the heating pad 1451E is not limited to a rectangular shape in a planar view. Any other shape may be possible if the heating pad 1451E can be elastically deformed in the thickness direction of the first flexible substrate 14E with the edges of the cut-away portions 1411, 1431 as fulcrums.

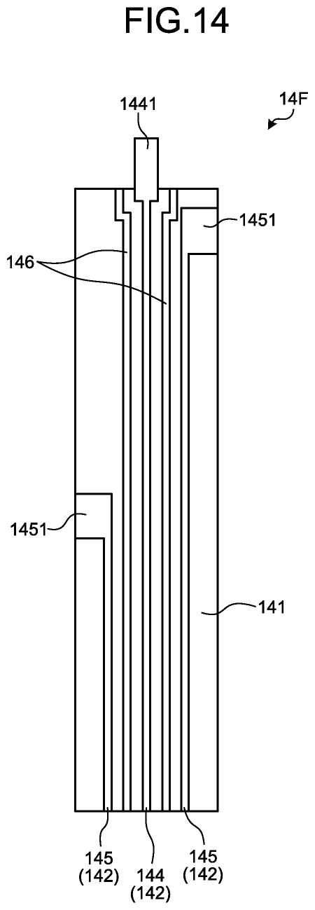

FIG. 14 is a view illustrating a sixth modification of the present embodiment. In FIG. 14, the second insulating layer 143 is omitted for convenience of description.

In the embodiment described above, a first flexible substrate 14F according to the present sixth modification illustrated in FIG. 14 may be used instead of the first flexible substrate 14.

Specifically, as illustrated in FIG. 14, the first flexible substrate 14F is different from the first flexible substrate 14 described above in that a ground conductive line 146 is disposed on one surface of the first insulating layer 141 so as to partition the space between the high-frequency conductive line 144 and each of the pair of heating conductive lines 145. A ground line (not illustrated) constituting the electric cable C is connected (bonded) to an end of each of the pair of ground conductive lines 146 on a proximal end side.

The sixth modification described above achieves the following effect in addition to effects similar to those of the embodiment described above.

Meanwhile, it is assumed that high-frequency power as power for heating is supplied to the pair of heating connectors 161 (a pair of heating conductive lines 145). In this case, since the high-frequency conductive line 144 and the pair of heating conductive lines 145 are arranged side by side, interference may occur between the high-frequency conductive line 144 and each of the pair of heating conductive lines 145. In the present sixth modification, the ground conductive line 146 is disposed so as to partition the space between the high-frequency conductive line 144 and each of the pair of heating conductive lines 145. For this reason, in the case described above, it is possible to reduce the interference generated between the high-frequency conductive line 144 and each of the pair of heating conductive lines 145.

When the first flexible substrate 14F is formed of a multilayer substrate, the high-frequency conductive line 144 and the pair of heating conductive lines 145 may be disposed on separate layers, and the ground conductive line 146 may be disposed as a solid pattern between the layer of the high-frequency conductive line 144 and the layer of the pair of heating conductive lines 145.

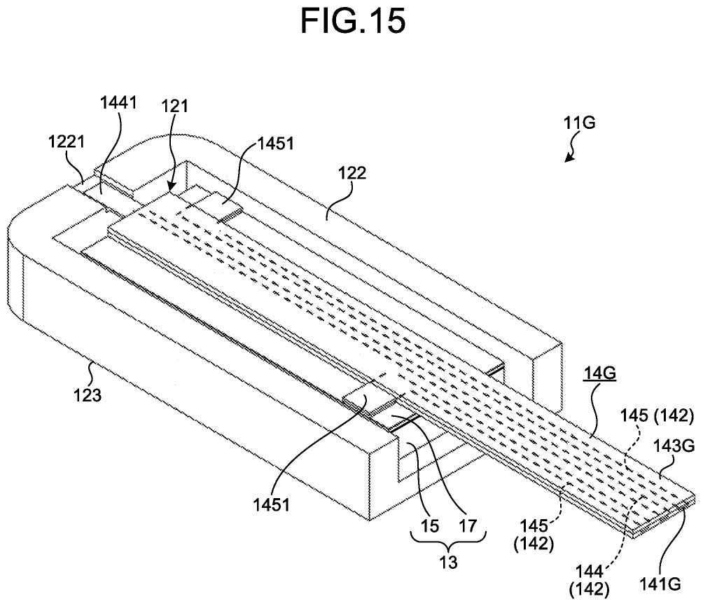

FIG. 15 is a view illustrating a seventh modification of the present embodiment.

In the embodiment described above, a first energy applying structure 11G according to the present seventh modification illustrated in FIG. 15 may be used instead of the first energy applying structure 11.

As illustrated in FIG. 15, the first energy applying structure 11G is different from the first energy applying structure 11 described in the above embodiment in that a flexible substrate 14G that includes first and second insulating layers 141G, 143G having a width dimension narrower than that of the first and second insulating layers 141, 143 is used instead of the first flexible substrate 14.

Specifically, the first and second insulating layers 141G, 143G have a shape in which both end portions of the first and second insulating layers 141, 143 described in the above embodiment in the width direction are omitted. As a result, the pair of heating pads 1451 project outward in the width direction from outer edges of the first insulating layer 141 and the second insulating layer 143.

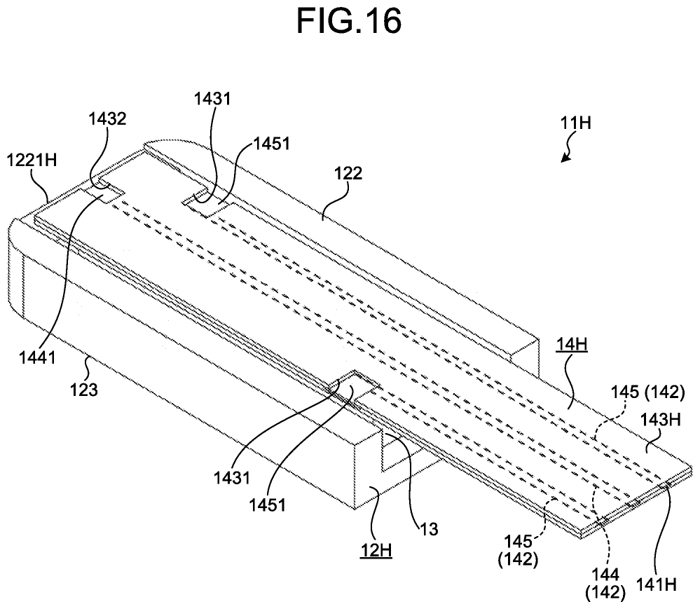

FIG. 16 is a view illustrating an eighth modification of the present embodiment.

In the embodiment described above, a first energy applying structure 11H according to the present eighth modification illustrated in FIG. 16 may be used instead of the first energy applying structure 11.

As illustrated in FIG. 16, the first energy applying structure 11H is different from the first energy applying structure 11 described in the above embodiment in that a flexible substrate 14H and a treatment tool 12H are used instead of the flexible substrate 14 and the treatment tool 12.

As illustrated in FIG. 16, the flexible substrate 14H is different from the flexible substrate 14 described in the above embodiment in that first and second insulating layers 141H, 143H having a longitudinal length dimension longer than that of the first and second insulating layers 141, 143 are used.

Specifically, in the first and second insulating layers 141H, 143H, ends on a distal end side substantially match the end of the high-frequency pad 1441 on the distal end side. Cut-away portions that respectively pass the first and second insulating layers 141H, 143H and expose a part of the high-frequency pad 1441 to outside are formed at positions facing the high-frequency pad 1441 on the first and second insulating layers 141H, 143H. FIG. 16 illustrates only a cut-away portion 1432 formed in the second insulating layer 143H among the cut-away portions formed in the first and second insulating layers 141H, 143H, respectively. The cut-away portions (cut-away portion 1432 in second insulating layer 143H) formed in the first and second insulating layers 141H, 143H, respectively correspond to an exposing aperture according to the present disclosure.

As illustrated in FIG. 16, the treatment tool 12H is different from the treatment tool 12 described in the above embodiment in that a bonding groove 1221H having a width dimension larger than that of the bonding groove 1221 is formed.

Specifically, the bonding groove 1221H cooperates with the change of the first and second insulating layers 141H, 143H described above. That is, the bonding groove 1221H has a width dimension slightly larger than the width dimension of the first and second insulating layers 141H, 143H, and allows the distal end side of the flexible substrate 14H to be disposed.

While the treatment device 2 according to the embodiment described above and the first to eighth modifications is configured to apply thermal energy and high-frequency energy to the living tissue LT, the present disclosure is not limited thereto. A configuration of further applying ultrasonic energy may be adopted.

In the treatment device 2 according to the embodiment described above and the first to eighth modifications, the first gripping surface 123 is configured as a flat surface, but the present disclosure is not limited thereto. For example, the first gripping surface 123 may be configured to have a projecting cross-sectional shape, a recessed cross-sectional shape, or a mountain-like cross-sectional shape. Similarly, the shape of the second gripping surface 191 is not limited to a mountain-like shape, and other shapes may be adopted.

In the embodiment described above and the first to eighth modifications, only the first gripping member 8 of the first gripping member 8 and the second gripping member 9 has a heating function. However, both the first and second gripping members 8 and 9 may have the heating function.

While the first energy applying structure 11 (11A to 11D) and the second energy applying structure 19 are electrically connected to the control device 3 by the electric cable C in the embodiment described above and the first to eighth modifications, the present disclosure is not limited thereto. For example, only the first flexible substrate 14 (14B, 14D to 14F) and the second flexible substrate 21 may electrically connect the first energy applying structure 11 (11A to 11D) and the second energy applying structure 19 to the control device 3.

The method of manufacturing a treatment device and the treatment device 2 according to the present embodiment achieve the effect that the assemblability is improved while compactness is achieved.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment device comprising:
a heater including
a first surface serving as a front surface,
a second surface serving as a back surface, and
a heating pattern formed on the first surface and configured to generate heat when energized;
a treatment tool bonded to the second surface and configured to apply high-frequency energy and thermal energy to a living tissue in contact with the treatment tool; and
a flexible substrate disposed so as to face the treatment tool with the heating member interposed between the flexible substrate and the treatment tool, the flexible substrate including
a first electrical conductor configured to supply high-frequency power to the treatment tool,
a second electrical conductor configured to supply power to the heating pattern, and
a pair of insulating layers facing each other with the first electrical conductor and the second electrical conductor interposed between the pair of insulating layers, wherein
the first electrical conductor includes a first bonding portion exposed from the pair of insulating layers and bonded to the treatment tool,
the second electrical conductor includes a second bonding portion exposed from the pair of insulating layers and bonded to the heating pattern,
the treatment tool includes a projection configured to project toward the flexible substrate and surround the heater, and
the first bonding portion is bonded to the projection.

2. The treatment device according to claim 1, wherein
each of the pair of insulating layers includes an exposing aperture configured to exposes at least one of the first bonding portion and the second bonding portion, and
at least one of the first bonding portion and the second bonding portion is exposed from the pair of insulating layers via the exposing aperture, faces a bonding target of at least one of the treatment tool and the heating pattern in a thickness direction of the flexible substrate, and is bonded to the bonding target.

3. The treatment device according to claim 2, wherein
the first bonding portion or the second bonding portion that is exposed from the pair of insulating layers via the exposing aperture is elastically deformable in the thickness direction of the flexible substrate with an edge of the exposing aperture as a fulcrum.

4. The treatment device according to claim 1, wherein
at least one of the first bonding portion and the second bonding portion projects from outer edges of the pair of insulating layers, faces a bonding target of at least one of the treatment tool and the heating pattern in a thickness direction of the flexible substrate, and is bonded to the bonding target.

5. The treatment device according to claim 1, wherein
the first bonding portion includes
a connecting portion configured to project from outer edges of the pair of insulating layers toward the treatment tool, and
a bonding portion main body that is bent from the connecting portion along an outer surface of the treatment tool, and
the bonding portion main body is configured to face the treatment tool in a thickness direction of the flexible substrate and bonded to the treatment tool.

6. The treatment device according to claim 1, wherein the second bonding portion is covered with an insulating resin.

7. The treatment device according to claim 1, wherein
a cutout is provided in the insulating layer of the flexible substrate, and
the second bonding portion is formed by exposing the second electrical conductor from the cutout.

8. A method of manufacturing a treatment device, the method comprising:
bonding a heater to a treatment tool so as to be surrounded by a projection formed on the treatment tool, the heater including a first surface with a heating pattern and a second surface, and the treatment tool applying high-frequency energy and thermal energy to a living tissue;
arranging a flexible substrate so as to face the treatment tool with the heating member interposed between the flexible substrate and the treatment tool; and
bonding the projection and a first bonding portion that is a first electrical conductor exposed from each of a pair of insulating layers of the flexible substrate.
